(19) United States
(12) Patent Application Publication
(10) Pub. No.: US 2009/0028934 A1
(43) Pub. Date: Jan. 29, 2009

(54) GRANULAR, GASTRO-RESISTANT PRODUCT BASED ON NIACIN OR DERIVATES THEREOF AND PROCESS FOR THE PRODUCTION OF SUCH A PRODUCT

(75) Inventor: Maurizio Lorenzon, Noale (IT)

Correspondence Address: CASTELLANO PLLC, P.O. Box 1555, Great Falls, VA, 22066, US

(73) Assignee: SILA s.r.l.

(21) Appl. No.: 12/095,292

(22) PCT Filed: Nov. 24, 2006
(86) PCT No.: PCT/EP06/11271

§ 371 Date: May 28, 2008

(30) Foreign Application Priority Data
Nov. 29, 2005 (IT) PD2005A000347

(51) Int. Cl. A61K 9/48; A61K 9/20; A61K 9/16; A61K 31/455; A61K 31/4406

(52) U.S. Cl. 424/451; 424/498; 424/464; 514/355; 514/356

(57) ABSTRACT

A granular, gastro-resistant product based on niacin or derivatives thereof comprises an internal part in which the niacin is substantially concentrated and an external coating, provided to cover and protect the internal part, made up of a matrix having a total weight fraction of long-chain C16-C22 saturated fatty acids of between 40% and 95% relative to the matrix. A process for the production of such a granular product is also described.

### TECHNICAL FIELD

The present invention relates to a granular, gastro-resistant product based on niacin or derivatives thereof according to the features set out in the precharacterising clause of main claim 1. It is also directed towards a process for the production of such a product having the features stated in the precharacterising clause of independent claim 11.

### BACKGROUND ART

Niacin, which is also known as vitamin B₃ or vitamin PP, is a substance which has very important metabolic functions in humans and animals, being a precursor of the coenzymes NAD and NADP which, in turn, are involved in the oxidation-reduction reactions (for example in the Krebs cycle) from which an organism obtains the energy it requires for its activities.

Strictly speaking, niacin corresponds to nicotinic acid, the structural formula of which is shown in (I), but the term is often used by analogy also to denote the amide of nicotinic acid, nicotinamide, the structural formula of which is shown in (II).

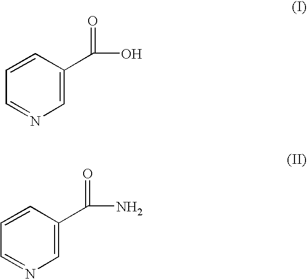

A deficiency of niacin in the organism results in an overall slowing of metabolism which is manifested, in the most serious cases, in the disorder known as pellagra. Niacin furthermore exhibits vasodilatory properties, for which reason it may be used for the therapeutic treatment of arterial hypertension, and is furthermore known to be effective in the control of high levels of cholesterol in the blood (hypercholesterolaemia). In particular, niacin is used for reducing the concentration of low-density cholesterol (LDL-C), known as “bad cholesterol” due to its direct involvement in cardiac disease.

The niacin required for correct functioning of the organism is largely obtained from outside the body, already in the form of NAD or NADP, in particular by intake of foods of animal origin, but if necessary it may also be taken as such, either in the form of nicotinic acid or in the form of nicotinamide.

It is, however, known that taking relatively large quantities of niacin may bring about the appearance of severe and unwanted skin redness similar to sunburn (a phenomenon known as “flushing”) which is often accompanied by pruritus, gastric disorders and a reduction in arterial pressure. This series of contraindications usually has very severe impact on individuals, such that, according to recent clinical studies, a very high proportion of patients, estimated to be between 10% and 50%, would give up treatment precisely because of these disadvantages.

In order to limit the contraindications associated with the administration of niacin, in particular the phenomenon of flushing, various solutions have been developed which aim to slow the release of niacin in the organism, in particular its release in the stomach. A first example of such solutions involves dispersing an appropriate quantity of niacin in a matrix of excipient substances, while a second known solution, described for example in U.S. Pat. No. 4,868,180, proposes encapsulating the niacin in a coating based on cellulosic substances.

Both U.S. Pat. No. 3,080,293 and No. 3,037,911, disclose a process for incorporating niacin in a saturated fatty acid matrix, in particular in a stearic acid matrix, in order to improve the disagreeable taste of the niacin as such. However, these solutions do not satisfactorily overcome the drawbacks encountered, for which reason a need subsists in this technical field to provide a niacin-based product which exhibits reduced side-effects, in particular which significantly reduces the phenomenon of flushing.

### DISCLOSURE OF THE INVENTION

The problem underlying the present invention is that of providing a granular, gastro-resistant product based on niacin or derivatives thereof and of providing a process for the production of such a product, these being structurally and functionally designed to overcome the above-stated shortcomings of the cited prior art.

In the context of this problem, a first object of the invention is furthermore to provide a granular product based on nicotinic acid in a form usable in the most varied applications in the field of human nutrition.

A second object is to provide a granular product with an elevated nicotinic acid content.

Said problem is solved and said objects are achieved by the present invention by means of a granular product and a process for the production of such a product in accordance with the following claims.

The features and advantages of the invention will emerge more clearly from the following detailed description in conjunction with a preferred embodiment thereof.

The product of the present invention is produced from niacin or derivatives thereof in granular form, which, in a first phase of the process, is uniformly mixed with a matrix of appropriate composition defined in greater detail below.

The mixing operation is carried out at a temperature such as to cause a large proportion of the components of the matrix to melt, but, at the same time, such as not to cause thermal degradation or impairment of the substances present in the mixture. This temperature is less than 120° C. and, preferably, is between 60° C. and 75° C., and, still more preferably, is around 70° C.

Mixing is carried out with a level of agitation and for a period of time which are sufficient to make the mixture as uniform and homogeneous as possible.

The granules of nicotinic acid, preferably in pure form, have a size of between 0.1 and 1.5 mm, which is also selected as a function of the desired mean size and size distribution for the finished product. The nicotinic acid granules are preferably between 0.1 and 0.5 mm in size.

According to a first aspect of the present invention, the matrix used in the mixture has a total content of long-chain C16-C22 saturated fatty acids of between 40% and 95%, relative to the matrix. In particular, it comprises a weight fraction of saturated C18 (stearic acid) of between 40% and 95%, preferably between 60% and 80%.

In this context, the total content of fatty acids is calculated taking account both of the presence of fatty acids as such and of the presence thereof in ester or salt form. In particular, the matrix comprises a component based on glycerides, which includes the above-stated C16-C22 fatty acids, and may comprise not only monoglycerides, but also diglycerides and triglycerides. Preferably, the fatty acid component associated with glycerides originates from triglycerides derived from vegetable oils (such as for example palm oil and sunflower oil) or from animal fats (such as for example lard or suet) with an elevated content of long-chain fatty acids. The triglycerides derived from these sources are appropriately hydrogenated to remove any double bonds optionally present in the chain, so as to give rise to saturated fatty acids in the above-stated percentages.

The matrix may optionally also comprises triglycerides having C12-C14 medium to short chain fatty acids, such as for example those derived from coconut oil and/or from soya oil, or, on the contrary, more complex longer chain aliphatic substances, such as for example waxes.

According to a second aspect of the invention, the matrix also contains mineral agents, such as for example calcium carbonate CaCO₃ or, preferably, calcium sulfate dihydrate CaSO₄*2(H₂O).

The mineral agent is present in the matrix in a weight fraction of between 2% and 20%. The presence of said mineral agent makes it possible to impart the correct consistency to the matrix either in the molten state or in the solid state and assists in ensuring that the finished product exhibits correct release kinetics of nicotinic acid in the organism.

The matrix furthermore contains an effective quantity of emulsifying agent, such as to make the matrix as uniform and homogeneous as possible in the molten state.

Suitable emulsifying agents comprise salts and esters of fatty acids, lecithins, esters of mono- and diglycerides, sugar esters of fatty acids, sorbitan-based esters and others.

Of course, should the emulsifying agent comprise a salt or an ester of a C16-C22 saturated fatty acid, this quantity, appropriately broken down, will be included in the overall calculation of the C16-C22 saturated fatty acids, with the aim of obtaining the above-stated weight fractions.

The matrix may furthermore contain further additives, such as for example natural flavours, in a variable concentration of between 0% and 2%.

The weight fraction of nicotinic acid granules mixed with the matrix is the highest fraction which permits the finished granular product to maintain its characteristics of slow release into the organism without giving rise to significant flushing phenomena. In particular, it has surprisingly been found that, by using a matrix produced according to the present invention, it is possible to obtain finished granular products complying with the above-stated requirements which comprise a weight fraction of nicotinic acid of approximately 70%.

However, to ensure a greater safety margin, it is preferred to add nicotinic acid granules to the matrix in a quantity such as to obtain a weight fraction of approximately 50%.

Once the desired level of homogeneity has been obtained, the mixture is supplied at high pressure to a spraying chamber where, by passing through an appropriately shaped jet, it is dispersed into a plurality of particles. The temperature in the spraying chamber is kept at values very much below the solidification temperature of the matrix, preferably of below −5° C., still more preferably at a temperature of between −20° C. and −25° C., such that, during the particles' brief residence time in the air, the liquid component thereof is conveniently able to solidify.

The size of the granules obtained from solidification of the biphasic particles sprayed into the chamber is a function of the shape and diameter of the spray jet, together with other parameters such as the velocity at which the mixture is sprayed, the viscosity thereof etc. Said parameters are appropriately adjusted such that the majority of granules are of a size within the desired range of between 0.15 and 2 mm, preferably of between 0.25 and 0.8 mm.

In any event, the product obtained is however subjected to a screening process which makes it possible to select the grain size which fulfils the required standards.

The finished product obtained exhibits a granular form and comprises an internal part made up of the original nicotinic acid granules and an external coating covering and protecting the former made up of the matrix which has solidified around the nicotinic acid granule. The nicotinic acid granule is thus microencapsulated in a coating of a mineral-lipid matrix.

Thanks to the specific composition of the mixture, in particular of the matrix, and to the above-described specific production process, the coating obtained is arranged continuously and uniformly around the internal part of niacin. This advantageously makes it possible to avoid (or at least greatly to limit) the presence of any surface outcrops of niacin which would constitute points permitting ready attack by gastric juices and would thus result in relatively rapid release of niacin into the organism.

The tests carried out on the granular product of the present invention, which are described in detail in the following paragraphs, have demonstrated excellent resistance to gastric attack, such as to enable gradual release of nicotinic acid along the entire gastrointestinal tract, so avoiding flushing phenomena in subjects taking the product. These results have been achieved even with granular products with a very high nicotinic acid content of greater than 40% and up to 70%.

The granular product according to the invention may be used, depending on dosage, both in the production of drugs and in the production of food supplements, which are in turn intended both for animals and for human beings.

In particular, the present granular product is preferably used as a food supplement for human beings, to be used in the preparation of solid or liquid food supplements, of beverages or solid foods, such as for example food bars, sweets, fruit juices, milk and derived products or bakery products.

In the pharmaceutical field, the microencapsulated niacin granules may be administered in the form of capsules, tablets, suspensions, etc.

### EXAMPLE

40 g of appropriately hydrogenated triglycerides obtained from sunflower oil with a high C18 content, 3 g of CaSO₄*2(H₂O), 2 g of CaCO₃ and 5 g of propylene glycol distearate (PGD) as emulsifying agent were placed in a mixer. The mixture was adjusted to a temperature of 70° C., so as to melt the lipid components and form a solid suspension in the liquid phase.

50 g of pure nicotinic acid in granular form, of a size between 0.1 and 0.5 mm, were added to said first mixture, which constitutes the matrix, while the mixture was constantly agitated.

The resultant mixture was then supplied to a spraying chamber kept at a temperature of approximately −22° C. where it was sprayed through a jet suited to the desired grain size, so as to obtain granules with an internal part based on niacin microencapsulated by an external coating based on a mineral-lipid matrix.

The granules taken from the spraying chamber were subjected to screening to obtain those granules of a size of between 0.25 and 0.8 mm.

### Product Analysis

Three samples of the granules obtained by the above-described process were subjected to in vitro testing to determine their behaviour along the human gastrointestinal tract, simulated by three distinct phases.

In the first phase, the samples were placed in respective 100 ml conical flasks to which were added 25 ml of a 0.1 M solution of phosphate buffer at pH 6 with gentle agitation, followed by 10 ml of a 0.2 M solution of HCl, the overall pH being adjusted to 2.0. 25 mg of pepsin in solution were then added to this solution and the overall mixture was kept for 2 hours in a controlled temperature environment at 39° C.

In the second phase, a phosphate buffer and 0.6 M NaOH were added to the mixture obtained from the first phase to adjust the overall pH to 6.8, and 100 mg of pancreatin in solution were added. The samples were then incubated for 4 hours at a temperature of 39° C.

In the third phase, the overall pH of the solution was adjusted to 7.0 by addition of 1 M NaOH, after which 100 mg of lipases, obtained from pig pancreas (SIGMA L-3126), were added and the solution was incubated for 18 hours at 39° C.

At the end of each of the three phases, portions of the three samples were taken and analysed by HPLC with the aim of ascertaining their nicotinic acid concentration.

The results are shown in Table 1 below.

TABLE 1

| | | After phase | After phase | After phase |
|---|---|---|---|---|
| | Beginning | 1 | 2 | 3 |
| Nicotinic acid | 53.56 | 44.32 | 34.17 | 2.72 |
| concentration (%) | | | | |
| Released nicotinic | 0 | 17.4 | 36.3 | 94.9 |
| acid (%) | | | | |

It is clear from examination of the results shown above that only very little release of niacin occurs during the time spent in the stomach and that, on the contrary, release predominantly occurs in the intestinal digestive phase due to the action of the enzymes. In this manner, virtually complete, entirely satisfactory release of the active ingredient is obtained, said release in particular being well distributed over time such that the concentration of niacin absorbed by the organism is never so high as to bring about flushing phenomena in the individual taking it.

The present invention thus solves the above-described problem with reference to the cited prior art and simultaneously provides further advantages including the production of a granular product based on microencapsulated nicotinic acid with an elevated content of active ingredient. This feature makes it possible to use a relatively limited quantity of finished granular product to administer the required quantity of nicotinic acid to the individual and proves to be particularly important when the product according to the present invention is to be used as a food supplement. It should be noted that the dosages which may be administered daily to individuals being treated with nicotinic acid are of the order of one gram.

A further advantage provided by the present invention is that of permitting safe handling of the product based on nicotinic acid without entailing special safety measures, nicotinic acid being well known to have irritant properties.

1. A granular, gastro-resistant product based on niacin or derivatives thereof, comprising an internal part in which said niacin or derivatives thereof is/are substantially concentrated and an external coating covering and protecting said internal part, said external coating comprising a matrix having a total weight fraction of long-chain C16-C22 saturated fatty acids of between 40% and 95% relative to the matrix, wherein said matrix further comprises a mineral agent, dispersed into the same, in a weight fraction of between 2% and 20% relative to the matrix.

2. A product according to claim 1, wherein said granular product has a size of between 0.15 and 2 millimetres.

3. A product according to claim 2, wherein said granular product has a size of between 0.25 and 0.8 millimetres.

4. A product according to claim 1, wherein said matrix comprises a weight fraction of C18 saturated fatty acid of between 40% and 95% relative to the matrix.

5. A product according to claim 4, wherein said weight fraction of C18 saturated fatty acid is between 60% and 80% relative to the matrix.

6. A product according to claim 1, wherein said mineral agent is calcium sulfate dihydrate.

7. A product according to claim 1, wherein said matrix comprises an effective quantity of emulsifying agent.

8. A product according to claim 7, wherein said emulsifying agent is based on propylene glycol distearate.

9. A product according to claim 1, wherein said niacin is present in a weight fraction of between 40% and 70%.

10. A granular, gastro-resistant product based on niacin or derivatives thereof according claim 1, for use in the treatment of arterial hypertension or hypercholesterolaemia.

11. A process for the production of granular gastro-resistant products based on niacin or derivatives thereof, comprising:
mixing granular material based on niacin or derivatives thereof with a matrix having a total content of long-chain C16-C22 saturated fatty acids of between 40% and 95% relative to the matrix,
adjusting said mixture to a temperature such as to melt said saturated fatty acids, and
spraying said mixture into a chamber with a temperature below the melting temperature of the matrix, such that said matrix solidifies around said granular material, so forming a product having an internal part in which said niacin or derivatives thereof is/are substantially concentrated and an external coating made up of said matrix,
wherein said matrix further comprises a mineral agent in a weight fraction of between 2% and 20% relative to the matrix.

12. A process according to claim 11, wherein said granular material based on niacin has a size of between 0.1 and 1.5 millimetres.

13. A process according to claim 12, wherein said granular material based on niacin has a size of between 0.1 and 0.5 millimetres.

14. A process according to claim 11, wherein said matrix comprises a weight fraction of C18 saturated fatty acid of between 40% and 95% relative to the matrix.

15. A process according to claim 14, wherein said weight fraction of C18 saturated fatty acid is between 60% and 80% relative to the matrix.

16. A process according to claim 11, wherein said mineral agent is calcium sulfate dihydrate.

17. A process according to claim 11, wherein an effective quantity of emulsifying agent is provided in said matrix.

18. A process according to claim 17, wherein said emulsifying agent is based on propylene glycol distearate.

19. A process according to claim 11, wherein said niacin is mixed with said matrix in a weight fraction of between 40% and 70% relative to the complete mixture.

20. A process according to claim 11, wherein said mixing phase is carried out at a temperature of between 60° C. and 75° C.

21. A process according to claim 11, wherein said mixture is sprayed into a chamber having a temperature of below −5° C.

22. A process according to claim 21, wherein said mixture is sprayed into a chamber having a temperature of between −20° C. and −25° C.

23. (canceled)

24. (canceled)

25. A method of treating arterial hypertension or hypercholesterolaemia in a mammal comprising administering a granular product as defined in claim 1 to the mammal.

26. The method of claim 25, wherein said granular product is administered to the mammal as a food supplement.

27. The method of claim 25, wherein said granular product is administered to the mammal by a route selected from the group consisting of capsules, tablets, and suspensions.

28. A food supplement for human beings comprising a granular product as defined in claim 1.
